(19) United States
(12) Patent Application Publication
(10) Pub. No.: US 2025/0134476 A1
(43) Pub. Date: May 1, 2025

(54) SYSTEMS, METHODS, AND FEATURES FOR ANALYTE MONITORING

(71) Applicant: ABBOTT DIABETES CARE INC., Alameda, CA (US)

(72) Inventors: William Koo Lee, Alameda, CA (US); Andres J. Palma Merino, Bogota D.C. (CO); Andrew M. Revoltar, Burien, WA (US); Chano Y. Puente de la Vega, San Francisco, CA (US); Jessica Rose Floeh, Brooklyn, NY (US); John Barry, Walnut Creek, CA (US); Jonathan M. Fern, Alameda, CA (US); Saranpreet S. Nagra, Alameda, CA (US); Taylor M. Charlesworth, Oakland, CA (US); Vaishnavi Ramesh, San Jose, CA (US)

(21) Appl. No.: 18/756,499

(22) Filed: Jun. 27, 2024

Related U.S. Application Data
(60) Provisional application No. 63/523,723, filed on Jun. 28, 2023.

(51) Int. Cl. A61B 5/00; G16H 40/67

(52) U.S. Cl. CPC A61B 5/7475; A61B 5/743; G16H 40/67

(57) ABSTRACT

Improved graphical user interfaces and wireless communication features for analyte monitoring software applications are provided. For example, disclosed herein are various embodiments of methods, systems, and interfaces for displaying data indicative of an analyte level for an analyte monitoring software application capable of receiving data according to more than one wireless communication protocol.

### CROSS-REFERENCE TO RELATED APPLICATIONS

This application claims priority to U.S. Provisional Application No. 63/523,723, filed Jun. 28, 2023, which is herein expressly incorporated by reference in its entirety for all purposes.

### FIELD

The subject matter described herein relates generally to digital interfaces, user interfaces, and features for analyte monitoring, including software applications, as well as systems, methods, and devices relating thereto.

### BACKGROUND

The detection and/or monitoring of analyte levels, such as glucose, ketones, lactate, oxygen, hemoglobin A1C, or the like, can be vitally important to the overall health of a person, particularly for an individual having diabetes. Patients suffering from diabetes mellitus can experience complications including loss of consciousness, cardiovascular disease, retinopathy, neuropathy, and nephropathy. Persons with diabetes are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with the bodily fluid. Furthermore, the benefits of analyte monitoring systems are not limited to persons with diabetes. For instance, analyte monitoring systems can provide useful information and insights to individuals interested in improving their health and wellness.

Despite their advantages, however, some people are reluctant to use analyte monitoring systems for various reasons, including the complexity and volume of data presented, a learning curve associated with the software and user interfaces for analyte monitoring systems, and an overall paucity of actionable information presented. In addition, analyte monitoring software applications on reader devices are sometimes susceptible to signal and communication failures, which can lead to decreased availability and reliability of analyte data.

Thus, needs exist for digital and graphical user interfaces and features for analyte monitoring systems and, in particular, analyte monitoring software applications, as well as methods and devices relating thereto, that are robust and capable of more reliable forms of data communication.

### SUMMARY

Provided herein are example embodiments of digital and user interfaces and features for analyte monitoring software applications. Aspects of the inventions are set out in the independent claims and preferred features are set out in the dependent claims. Preferred features of each aspect may be provided in combination with each other within particular embodiments and may also be provided in combination with other aspects. Improved graphical user interfaces and wireless communication features for analyte monitoring software applications are provided. For example, disclosed herein are various embodiments of methods, systems, and interfaces for displaying data indicative of an analyte level for an analyte monitoring software application capable of receiving data according to more than one wireless communication protocol.

According to some embodiments, systems, methods, and graphical user interfaces for an analyte monitoring software application that is capable of receiving data indicative of an analyte level from a sensor control device via more than one wireless communication protocol are provided.

According to some embodiments, systems, methods, and graphical user interfaces for an analyte monitoring software application having an on-demand data backfilling feature are provided, wherein the analyte monitoring software application is capable of receiving data indicative of an analyte level from a sensor control device via more than one wireless communication protocol. In some embodiments, said analyte monitoring software applications can also include a feature to calculate and display projected missing historical analyte data.

According to another embodiment, systems, methods, and graphical user interfaces for an analyte monitoring software application having a logbook feature are provided, wherein the analyte monitoring software application is capable of receiving data indicative of an analyte level from a sensor control device via more than one wireless communication protocol.

According to some embodiments, systems, methods, and graphical user interfaces are provided for enabling a new or upgraded feature or functionality in an analyte monitoring software application. In addition, according to some embodiments, a method of dynamic transmission rates between an analyte monitoring software application and a trusted computer system are provided.

Many of the embodiments provided herein improve upon wireless communications within a continuous glucose monitoring system. In particular, the use of more than one wireless communication protocol between an analyte monitoring software application on a reader device and the sensor control device can mitigate a single point-of-failure, reduce latency, and alleviate other problems that can arise especially in harsh signal environments.

Furthermore, many of the embodiments provided herein are improved GUIs or GUI features for analyte monitoring systems that are highly intuitive, user-friendly, and provide for rapid access to physiological information of a user. More specifically, these embodiments allow a user to easily navigate through and between different user interfaces that can quickly indicate to the user various physiological conditions and/or actionable responses, without requiring the user (or an HCP) to go through the arduous task of examining large volumes of analyte data. The various configurations of these devices are described in detail by way of the embodiments which are only examples.

Other systems, devices, methods, features and advantages of the subject matter described herein will be or will become apparent to one with skill in the art upon examination of the following figures and detailed description. Where a method is described and claimed herein, analyte monitoring systems comprising means for performing each of the steps of the method are also expressly disclosed and provided. Moreover, computer programs, computer program products and computer readable media for implementing the steps of the method are also disclosed and provided. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIGS. 3A to 3D are example embodiments of GUIs for displaying data indicative of an analyte level for an analyte monitoring software application.

FIGS. 4A to 4D are example embodiments of GUIs for displaying data indicative of an analyte level for an analyte monitoring software application capable of receiving data according to more than one wireless communication protocol.

FIGS. 4E to 4G are flow diagrams depicting example embodiments of methods for displaying data indicative of an analyte level in an analyte monitoring software application capable of receiving data according to more than one wireless communication protocol.

FIGS. 5A to 5D are example embodiments of GUIs for displaying missing data indicative of an analyte level for an analyte monitoring software application capable of receiving data according to more than one wireless communication protocol.

FIG. 5E is a flow diagram depicting an example embodiment of a method for displaying missing data indicative of an analyte level in an analyte monitoring software application capable of receiving data according to more than one wireless communication protocol.

FIGS. 5F and 5G are example embodiments of GUIs for displaying projected data indicative of an analyte level for an analyte monitoring software application.

FIG. 5H is a flow diagram depicting an example embodiment of a method for displaying projected data indicative of analyte level for an analyte monitoring software application.

FIGS. 5I to 5K are example embodiments of GUIs for displaying error messages relating to data indicative of an analyte level for an analyte monitoring software application.

FIG. 6A is a flow diagram depicting an example embodiment of a method for enabling a new functionality of an analyte monitoring software application.

FIGS. 6B to 6G are example embodiments of GUIs relating to a method for enabling a new functionality of an analyte monitoring software application.

FIG. 7A is a flow diagram depicting an example embodiment of a method for displaying events in a logbook interface of an analyte monitoring software application capable of receiving data according to more than one wireless communication protocol.

FIGS. 7B to 7D are example embodiments of GUIs for displaying events in a logbook interface of an analyte monitoring software application capable of receiving data according to more than one wireless communication protocol.

FIG. 8 is a flow diagram depicting an example embodiment of a method for a dynamic transmission rate feature of an analyte monitoring software application.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include features, GUIs and digital interfaces for analyte monitoring systems, analyte monitoring software applications, and systems, methods, and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

Collectively and individually, these methods, systems, and digital and user interfaces improve upon the accuracy, integrity, and reliability of analyte data being collected by an analyte monitoring system. Other improvements and advantages are provided as well. The various configurations of these devices are described in detail by way of the embodiments which are only examples.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

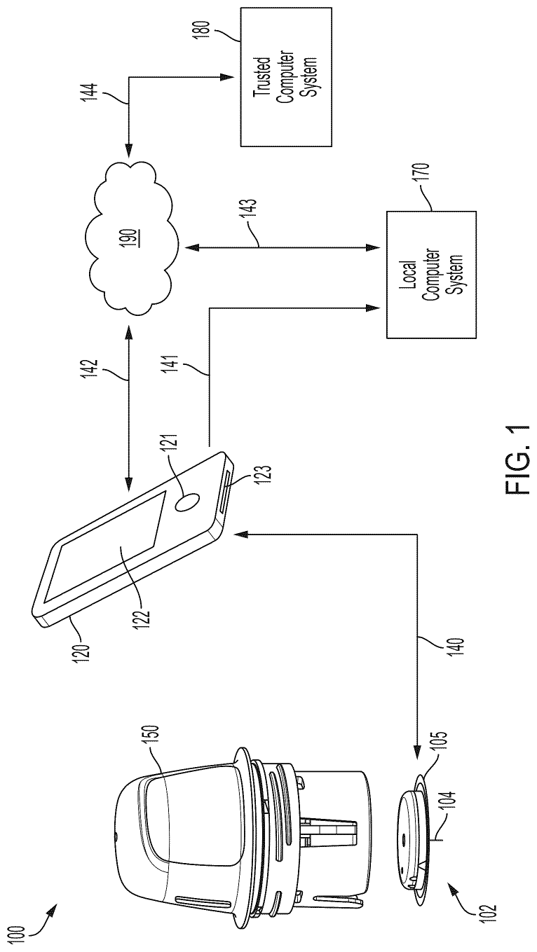

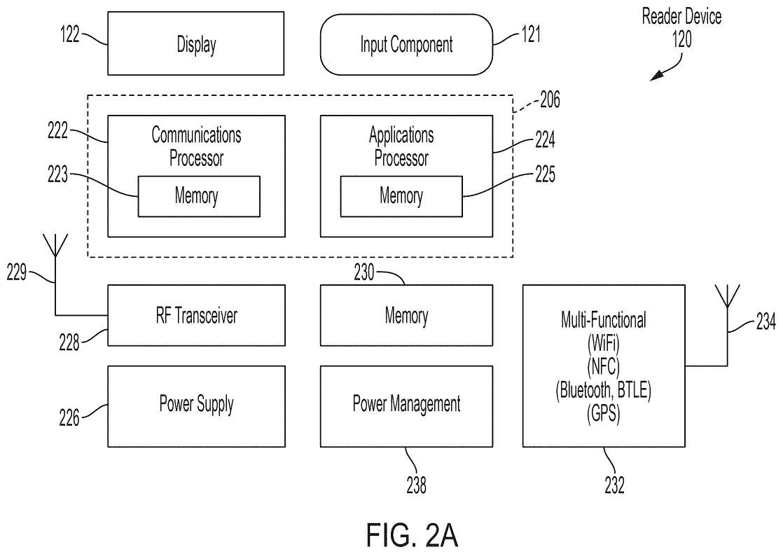

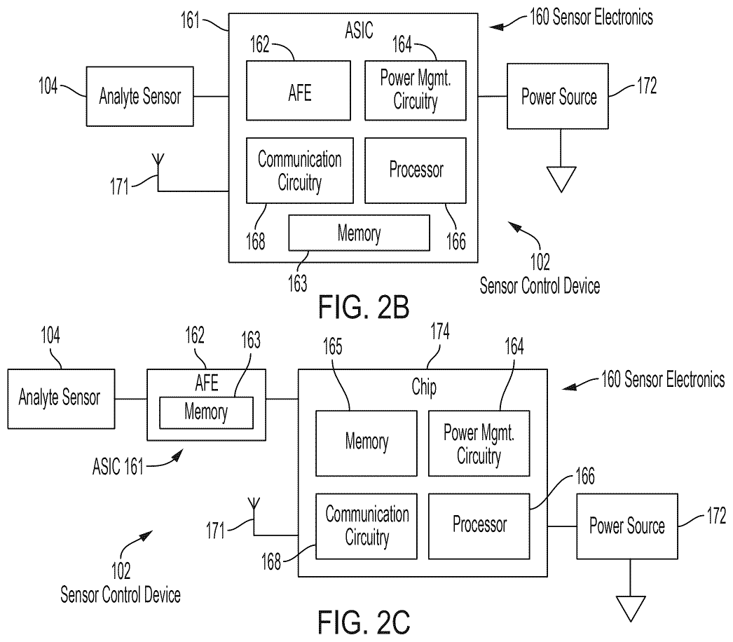

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 are set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a cloud-based platform or server, and can provide for authentication services, secured data storage, report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices.

### Example Embodiment of Reader Device

FIG. 2A is a block diagram depicting an example embodiment of a reader device 120, which, in some embodiments, can comprise a smart phone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical glucose values can be transmitted from sensor control device 102 to reader device 120 every five minutes.

In some embodiments, data acquired from sensor control device 102 can be stored on reader device 120. According to one aspect of some embodiments, such data can include the model number and serial number of sensor control device 102, as well as information relating to the sensor control device 102's status, market code, or network address. In some embodiments, such data can also include error events detected by sensor control device 102. In addition, in some embodiments, either or both of current glucose values and historical glucose values can include one or more time stamps (e.g., factory time, UTC time, user's local time based on time zone, and the current time zone).

In some embodiments, sensor control device 102 can store data such that if reader device 120 is not in communication with sensor control device 102 (e.g., if reader device 120 is out of a wireless communication range, is powered off, or is otherwise unable to communicate with sensor control device 102), when reader device 120 re-establishes communication with sensor control device 102, data can then be backfilled to reader device 120. According to some embodiments, data that can be backfilled can include, but is not limited to, current and historical glucose values, as well as error events. Further details regarding data backfilling can be found in U.S. Pat. No. 10,820,842, as well as U.S. Publ. No. 2021/0282672 (“the '672 Publication”), both of which are hereby incorporated by reference in their entireties for all purposes.

According to some embodiments, each current glucose value and/or historical glucose value acquired from sensor control device 102 can further be validated on reader device 120, such as, for example, by performing a CRC integrity check to ensure that the data has been transferred accurately. In some embodiments, for example, a data quality mask of the current glucose value and/or historical glucose value can be checked to ensure that the reading is correct and can be displayed as a valid reading on the reader device 120.

According to another aspect of some embodiments, reader device 120 can include a database for storing any or all of the aforementioned data. In some embodiments, the database can be configured to retain data for a predetermined period of time (e.g., 30 days, 60 days, 90 days, six months, one year, etc.). According to some embodiments, the database can be configured to delete data after it has been uploaded to a cloud server. In other embodiments, database can be configured for a clinical setting, in which data is retained for a longer period of time (e.g., one year) relative to a non-clinical setting. In addition to the aforementioned data (e.g., current and/or historical glucose values, error events, etc.), the database on reader device 120 can also store user configuration information (e.g., login ID, notification settings, regional settings, and other preferences), as well as application configuration information (e.g., cloud settings, URLs for uploading data and/or error events, version information, etc.). The database can be encrypted to prevent a user from inspecting the data content directly even if the operating system of reader device 120 is compromised.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed—in whole or in part—by processing circuitry on sensor control device 102, reader device 120, local computer system 170, or trusted computer system 180.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Analyte Monitoring Software Applications

Described herein are example embodiments of analyte monitoring software applications for analyte monitoring systems, as well as methods and systems relating thereto. As an initial matter, it will be understood by those of skill in the art that the GUIs and related methods described herein comprise instructions stored in a non-transitory memory of reader device 120, local computer system 170, trusted computer system 180, and/or any other device or system that is part of, or in communication with, analyte monitoring system 100. These instructions, when executed by one or more processors of the reader device 120, local computer system 170, trusted computer system 180, or other device or system of analyte monitoring system 100, cause the one or more processors to perform the method steps and/or output the GUIs described herein. Those of skill in the art will further recognize that the GUIs described herein can be stored as instructions in the non-transitory memory of a single centralized device or, in the alternative, can be distributed across multiple discrete devices in geographically dispersed locations.

### Example Embodiments of Home Screen GUIs for an Analyte Monitoring Software Application

Example embodiments of graphical user interfaces, methods and systems relating thereto for displaying data indicative of an analyte level received from a sensor control device on a home screen of an analyte monitoring software application will now be described. It will be understood by those of skill in the art that any one or more of the example embodiments of the methods, interfaces, and systems described herein can either be implemented independently, or in combination with any of the other embodiments described in the present application.

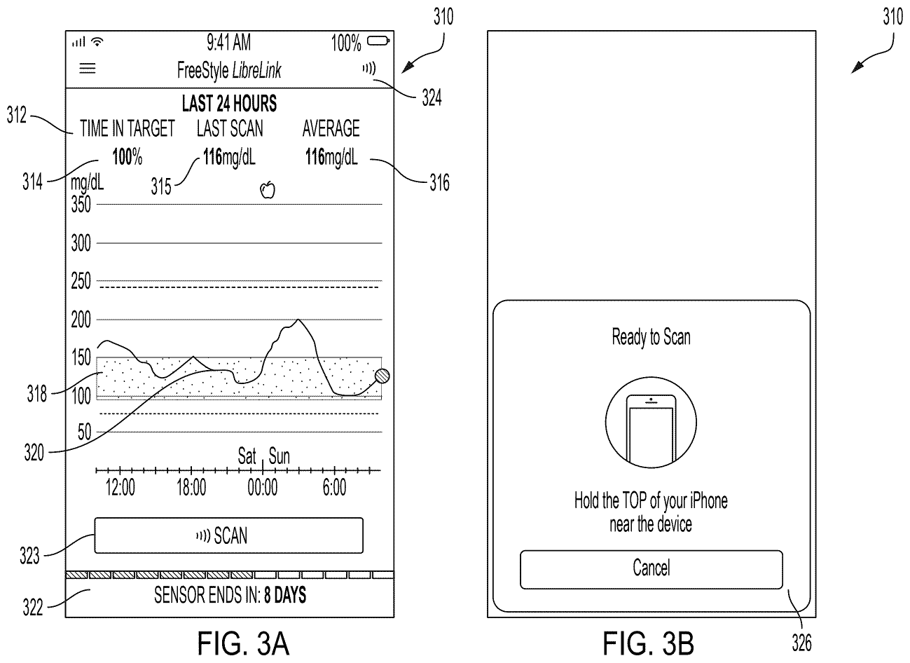

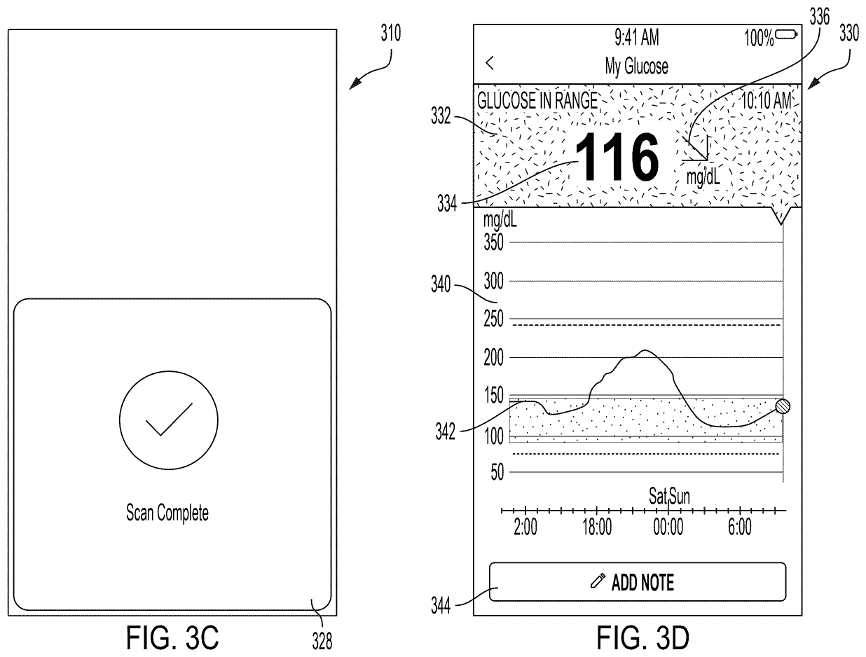

FIGS. 3A to 3D are example embodiments of GUIs for displaying data indicative of an analyte level in an analyte monitoring software application. According to one aspect of the example embodiments of FIGS. 3A to 3D, a sensor control device is configured to wirelessly transmit data indicative of an analyte level to a reader device according to a single wireless communication protocol. In some embodiments, for example, the wireless communication protocol can be a Near Field Communication (“NFC”) protocol. According to another aspect of the example embodiments of FIGS. 3A to 3D, the received data indicative of the analyte level can be displayed by an analyte monitoring software app on the reader device in various forms, as will be described in further detail.

For example, referring to FIG. 3A, a home screen GUI 310 for an analyte monitoring software application can comprise a banner section 312 that includes a time-in-target value 314 (e.g., a percentage of time that the analyte level is within a target analyte range), a last received analyte level value 315, and an average analyte level value 316. In some embodiments, home screen GUI 310 can further comprise an analyte trend graph section 318, including an analyte trend line 320 depicting a user's historical analyte levels over time. In some embodiments, home screen GUI 310 can also include a sensor expiration indicator 322, which is configured to display the remaining operating life of a current sensor or sensor control device. According to another aspect of some embodiments, home screen GUI 310 comprises a “scan” button 323 that, when actuated by a user, transmits a request to the sensor control device for data indicative of the analyte level. In some embodiments, home screen GUI 310 can also have a “scan” icon 324 which is also configured to transmit, when actuated by the user, a request to the sensor control device for data indicative of the analyte level.

According to one aspect of some example embodiments of FIGS. 3A to 3D, when scan button 323 or scan icon 324 is actuated, a series of informational modals can be displayed over home screen GUI 310. For example, as shown in FIG. 3B, a ready-to-scan modal 326 can indicate to the user how to position the reader device in relation to the sensor control device to initiate the request for data indicative of the analyte level. Similarly, as shown in FIG. 3C, a scan complete modal 328 can indicate to the user that the wireless transmission of data indicative of the analyte level from the sensor control device to the reader device, in response to the user-initiated request, is complete.

According to another aspect of some example embodiments of FIGS. 3A to 3D, after the data indicative of the analyte level has been transmitted from the sensor control device to the reader device via the wireless communication protocol, a sensor results GUI 330, as shown in FIG. 3D, can be displayed. In some embodiments, sensor results GUI 330 can comprise a banner section 332, which can include a current analyte level 334 (shown in numeric format) along with a trend arrow 336 to indicate the directional trend of the analyte level. According to some embodiments, sensor results GUI 330 can also include an analyte trend graph section 340, including an analyte trend line 342 depicting the user's historical analyte levels over time. With respect to banner section 332, current analyte level 334 and trend arrow 336 can reflect the most recently received data indicative of the analyte level transmitted from the sensor control device to the reader device. In some embodiments, GUI 330 can further comprise an “Add Note button 344 to allow the user to store an event into memory associated with all or a portion of the analyte trend line 342.

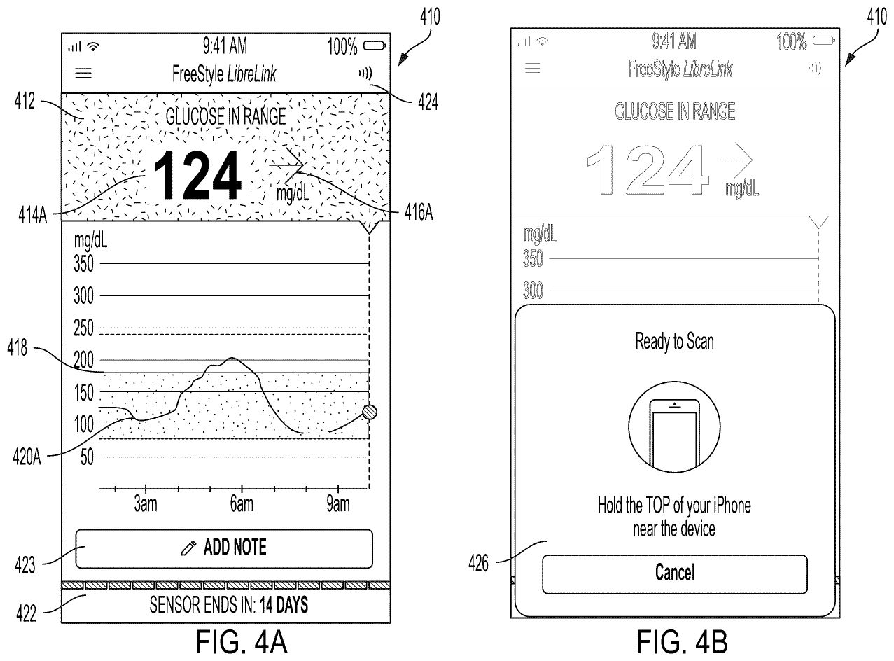

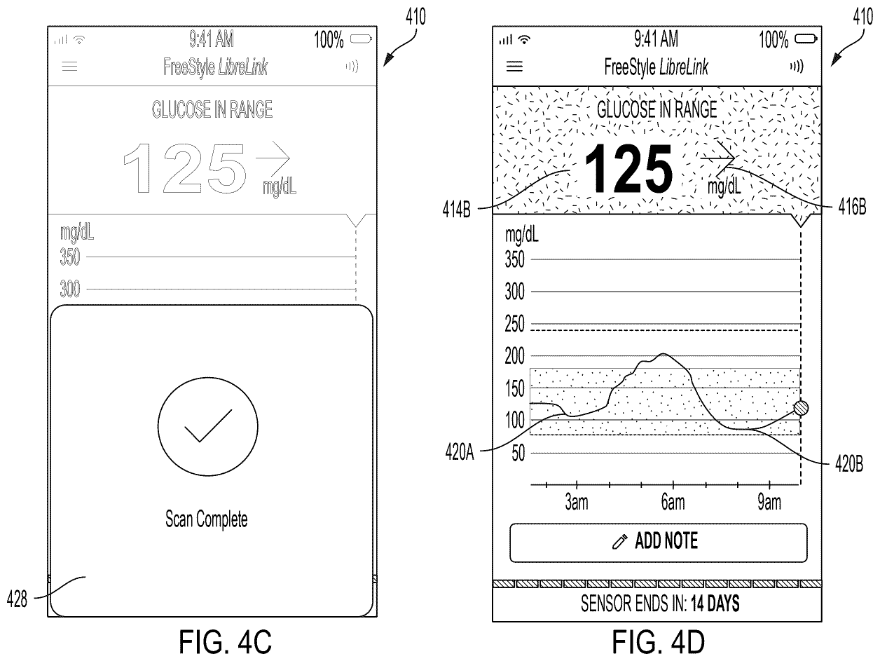

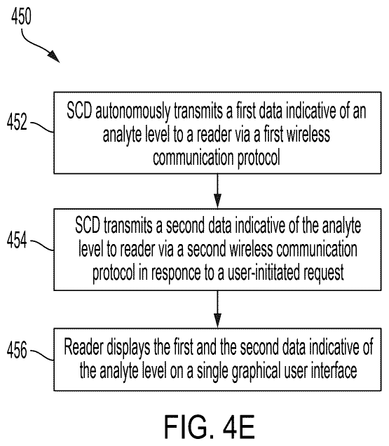

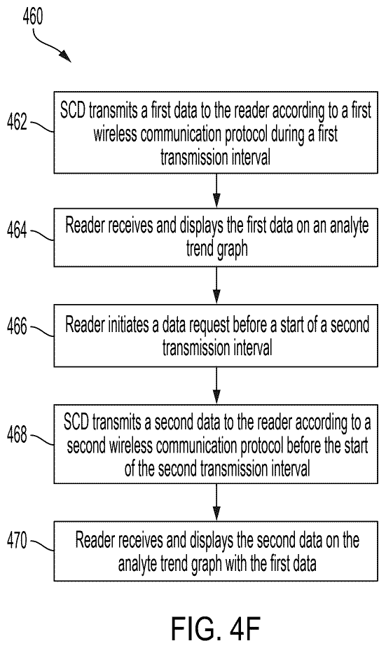

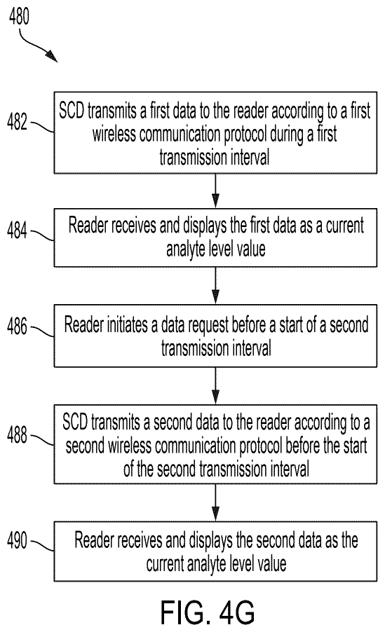

FIGS. 4A to 4D depict another set of example embodiments of GUIs for displaying data indicative of an analyte level in an analyte monitoring software application. In a general sense, the embodiments of FIGS. 4A to 4D share similarities with those of FIGS. 3A to 3D, except that the analyte monitoring software applications of FIGS. 4A to 4D are capable of receiving data indicative of a user's analyte levels according to more than one wireless communication protocol. In some embodiments, for example, the first wireless communication protocol can be a Bluetooth or Bluetooth Low Energy protocol, and the second wireless communication protocol can be an NFC protocol. Those of skill in the art will appreciate that other wireless communication protocols (e.g., cellular, 802.11x, infrared, UHF, etc.) can be utilized in combination with any of the embodiments described herein, and are within the scope of the present disclosure.

Referring now to FIG. 4A, a home screen GUI 410 for an analyte monitoring software application is depicted, wherein home screen GUI 410 comprises a banner section 412 having a current analyte level 414A (shown in numeric format) along with a trend arrow 416A to indicate the directional trend of the analyte level. According to many embodiments, home screen GUI 410 can also include an analyte trend graph section 418, including an analyte trend line 420A that depicts the user's historical analyte levels over time. With respect to banner section 412, current analyte level 414A and trend arrow 416A can reflect the most recently received data indicative of the analyte level transmitted from the sensor control device to the reader. In some embodiments, GUI 410 can further comprise an “Add Note” button 422 to allow the user to store an event into memory associated with all or a portion of the analyte trend line 420A.

According to one aspect of the example embodiments of FIGS. 4A to 4D, at least the analyte trend line 420A, current analyte level 414A, and trend arrow 416A, as shown in FIG. 4A, reflect data indicative of the user's analyte level received via a first wireless communication protocol from the sensor control device. As described earlier, in some embodiments, the first wireless communication protocol can be a Bluetooth or Bluetooth Low Energy protocol.

According to another aspect of the example embodiments of FIGS. 4A to 4D, home screen GUI 410 comprises a scan icon 424 that, when actuated by the user, can initiate a new request for data indicative of the user's analyte level to be transmitted from the sensor control device to the reader device via a second wireless communication protocol. As shown in FIGS. 4B and 4C, actuation of the scan icon 424 can cause the analyte monitoring software application to display information modals 426 and 428 to facilitate the transfer of data indicative of the user's analyte level from the sensor control device to the reader device via the second wireless communication protocol. As described earlier, in some embodiments, the second wireless communication protocol can be an NFC protocol.

Referring next to FIG. 4D, an example embodiment of home screen GUI 410 is depicted after receiving the data indicative of the user's analyte levels from the sensor control device via the second wireless communication protocol. According to some embodiments, current analyte level 414B and trend arrow 416B have replaced, respectively, the previous current analyte level 414A and trend arrow 416A (previously shown in FIG. 4A). In addition, according to some embodiments, analyte trend line 420B is displayed along with analyte trend line 420A (previously shown in FIG. 4A) to present a more complete record of the user's historical analyte levels over time.

One of the several advantages of the embodiments described with respect to FIGS. 4A to 4D is that data indicative of the user's analyte levels can be automatically sent from the sensor control device to the reader device and displayed in an analyte monitoring software application via a first wireless communication protocol (e.g., Bluetooth) without user intervention. In certain cases, however, a user may wish to immediately request their analyte data at a point in time between the transmission intervals of the first wireless communication protocol. In that scenario, a user may initiate an “on demand” transmission, e.g., by actuating the scan button 424 of home GUI 410, to request the data indicative of the user's analyte level to be sent by the sensor control device via a second wireless communication protocol (e.g., NFC).

Turning next to FIGS. 4E to 4G, example embodiments of methods for displaying data indicative of a user's analyte levels in a graphical user interface of an analyte monitoring software application will now be described, wherein the analyte monitoring software application is capable of receiving data via more than one wireless communication protocol. Those of skill in the art will appreciate that the methods described herein can comprise instructions stored in memory of a reader device, and further can comprise either a portion of, or all of, an analyte monitoring software application, such as the example embodiments described with respect to FIGS. 4A to 4D.

FIG. 4E depicts an example embodiment of a method 450 for displaying data indicative of a user's analyte levels in an analyte monitoring software application capable of receiving analyte data via more than one wireless communication protocol. At Step 452, a sensor control device (“SCD”) transmits a first data indicative of an analyte level to a reader device via a first wireless communication protocol. In some embodiments, the transmission can occur autonomously, e.g., without a request being sent from the reader to the sensor control device. For example, in some embodiments, as mentioned earlier, the first wireless communication protocol can be a Bluetooth or Bluetooth Low Energy protocol. At Step 454, the SCD transmits a second data indicative of the analyte level to the reader device via a second wireless communication protocol in response to a user-initiated request. In some embodiments, the user-initiated request can be, for example, the user actuating a scan button on a home screen GUI, as described with respect to FIGS. 4A to 4D. In other embodiments, the user-initiated request can be the user positioning the reader in close proximity to the sensor control device. In still other embodiments, the user-initiated request can be moving the reader according to a predetermined gesture or motion that can be detected by one or more motion sensors, positional sensors, or accelerometers disposed in the reader device. As mentioned earlier, the second wireless communication protocol can be, for example, an NFC protocol.

Referring still to FIG. 4E, at Step 456, the reader displays both the first data indicative of the analyte data and the second data indicative of the analyte data on a single graphical user interface (GUI) of the analyte monitoring software application. As described with respect to FIGS. 4A to 4D, in some embodiments, the first data can be a first portion 420A of an analyte trend line and the second data can be a second portion 420B of the analyte trend line. In some embodiments, the second data can be a current analyte level 414B or a trend arrow 416B. As such, a second wireless communication protocol can be utilized by a user to request analyte data “on demand” to supplement analyte data received via the first wireless communication protocol for an analyte monitoring software application. In this regard, this method can be advantageous for mitigating a single point-of-failure in wireless data communications and, relatedly, can also mitigate problems arising in harsh signal environments (e.g., interference from other devices).

FIG. 4F depicts another example embodiment of a method 460 for displaying data indicative of a user's analyte levels in an analyte monitoring software application capable of receiving analyte data via more than one wireless communication protocol. At Step 462, a sensor control device transmits a first data indicative of the user's analyte levels to the reader device according to a first wireless communication protocol during a first transmission interval. In some embodiments, for example, the transmission interval can be every minute, every two (2) minutes, every five (5) minutes, or every ten (10) minutes. Those of skill in the art will recognize that other fixed or variable rates can be utilized and are within the scope of the present disclosure. In some embodiments, the sensor control device can be configured to begin the transmission of the first data at a start of the transmission interval. In other embodiments, the transmission of the first data can be repeated multiple times during the transmission interval.

Referring still to FIG. 4F, at Step 464, the reader device receives the first data indicative of the user's analyte level and displays the first data on an analyte trend graph, such as trend graph 418 described with respect to FIG. 4A.

Next, at Step 466, the reader device initiates a request for data indicative of the user's analyte levels before the start of a second transmission interval. According to some embodiments, for example, the initiation of the request can occur in response to the user actuating a scan icon, such as the scan icon 422 that is described with respect to FIG. 4A. In other embodiments, the initiation of the request can occur in response to the user positioning the reader in close proximity to the sensor control device. In still other embodiments, the initiation of the data request can occur in response to a predetermined gesture or motion detected by the reader device. According to another aspect of some embodiments, the initiation of the request can occur during the first transmission interval. In other embodiments, the initiation of the data request can occur during an intervening period between the first transmission interval and a second transmission interval.

At Step 468, in response to receiving the request from the reader device, the sensor control device transmits a second data indicative of an analyte level of the user to the reader device using a second wireless communication protocol, wherein the transmission of the second data occurs before the start of a second transmission interval associated with the first wireless communication protocol. In some embodiments, for example, the second wireless communication protocol can be an NFC protocol. In some embodiments, the transmission of the second data indicative of the analyte level can occur during the first transmission interval or before the first transmission interval has ended. In other embodiments, the transmission of the second data can occur during an intervening period between the first transmission and the second transmission.

Referring still to FIG. 4F, at Step 470, the reader device receives the second data indicative of the user's analyte levels via the second wireless communication protocol. Subsequently, the second data is displayed in an analyte trend graph of the analyte monitoring software application with the first data indicative of the user's analyte level. For example, as shown in FIG. 4D, a second portion 420B of an analyte trend line is based on the second data received from the sensor control device according to a second wireless communication protocol. The second portion 420B of the analyte trend line is displayed on the same analyte trend graph as a first portion 420A of the analyte trend line, which is based on the first data received from the sensor control device according to the first wireless communication protocol.

FIG. 4G depicts another example embodiment of a method 480 for displaying data indicative of a user's analyte levels in an analyte monitoring software application capable of receiving analyte data via more than one wireless communication protocol. At Step 482, a sensor control device transmits a first data indicative of the user's analyte levels to the reader device according to a first wireless communication protocol during a first transmission interval. In some embodiments, for example, the transmission interval can be every minute, every two (2) minutes, every five (5) minutes, or every ten (10) minutes. Those of skill in the art will recognize that other fixed or variable rates can be utilized and are within the scope of the present disclosure. In some embodiments, the sensor control device can be configured to begin the transmission of the first data at a start of the transmission interval. In other embodiments, the transmission of the first data can be repeated multiple times during the transmission interval.

Referring still to FIG. 4G, at Step 484, the reader receives the first data indicative of the user's analyte level and displays the first data as a current analyte level value, such as current analyte level 414A, as shown in FIG. 4A.

Next, at Step 486, the reader device initiates a request for data indicative of the user's analyte levels before the start of a second transmission interval. According to some embodiments, for example, the initiation of the request can occur in response to the user actuating a scan icon, such as the scan icon 422 that is described with respect to FIG. 4A. In other embodiments, the initiation of the request can occur in response to the user positioning the reader in close proximity to the sensor control device. In still other embodiments, the initiation of the data request can occur in response to a predetermined gesture or motion detected by the reader device. According to another aspect of some embodiments, the initiation of the request can occur during the first transmission interval. In other embodiments, the initiation of the data request can occur during an intervening period between the first transmission interval and a second transmission interval.

At Step 488, in response to receiving the request from the reader device, the sensor control device transmits a second data indicative of an analyte level of the user to the reader device using a second wireless communication protocol, wherein the transmission of the second data occurs before the start of a second transmission interval associated with the first wireless communication protocol. In some embodiments, for example, the second wireless communication protocol can be an NFC protocol. Moreover, in some embodiments, the transmission of the second data indicative of the analyte level can occur during the first transmission interval or before the first transmission interval has ended. In other embodiments, the transmission of the second data can occur during an intervening period between the first transmission and the second transmission.

Referring still to FIG. 4G, at Step 490, the reader device receives the second data indicative of the user's analyte levels via the second wireless communication protocol. Subsequently, the second data is displayed as a current analyte level, such as current analyte level 414B (as shown in FIG. 4D), in replacement of the previous current analyte level described at Step 484. Similarly, a current analyte trend arrow, such as trend arrow 416B (as shown in FIG. 4D) can be displayed in replacement of the previous trend arrow 416A (as shown in FIG. 4A). In this manner, the user can always request a current analyte level value “on demand” via the second wireless communication protocol without having to wait for the second transmission interval to begin to obtain data via the first wireless communication protocol.

### Example Embodiments of On-Demand Analyte Data Backfilling Features and GUIs

Example embodiments of graphical user interfaces, methods and systems relating thereto for an analyte data backfilling feature of an analyte monitoring software application will now be described. It will be understood by those of skill in the art that any one or more of the example embodiments of the methods, interfaces, and systems described herein can either be implemented independently, or in combination with any of the other embodiments described in the present application.

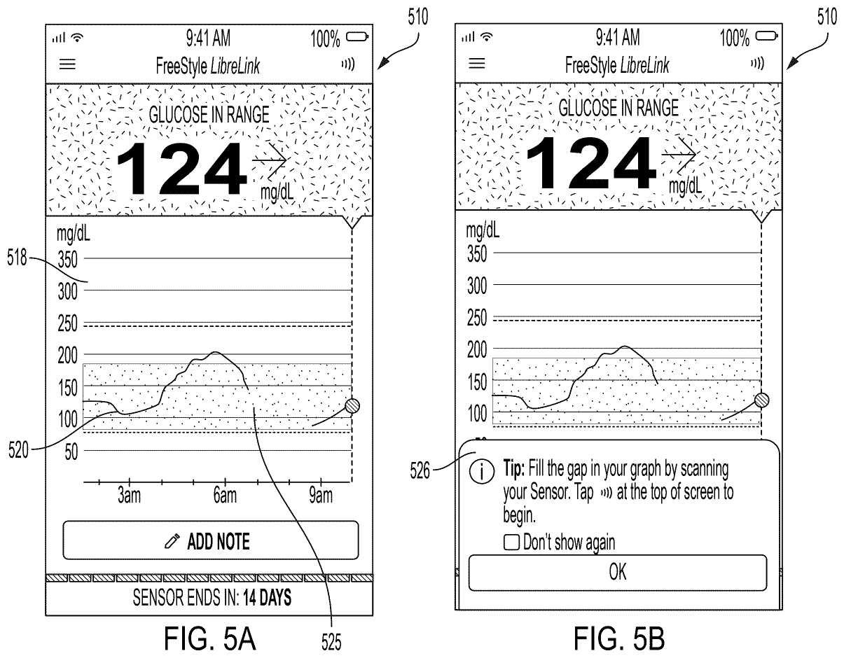

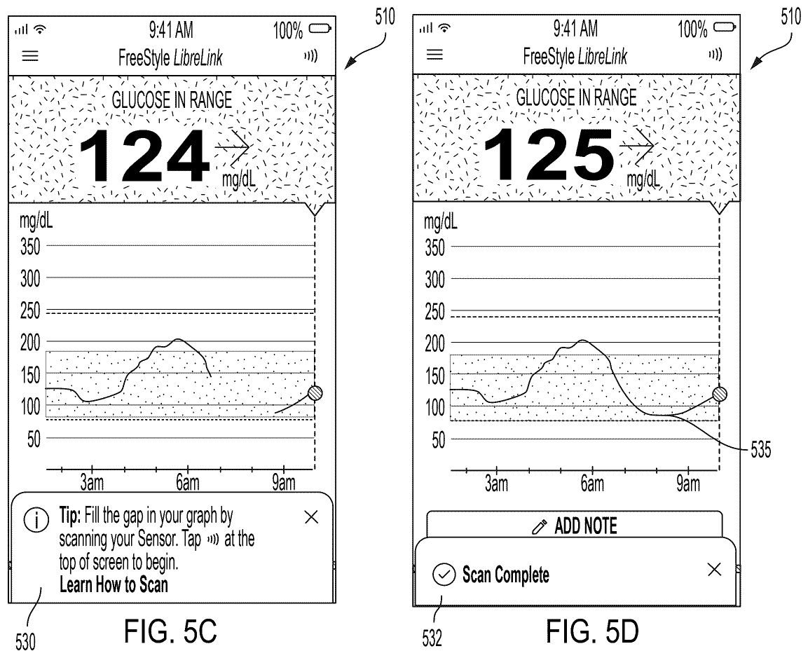

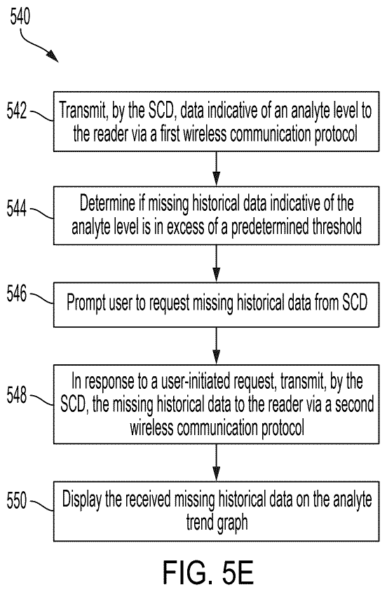

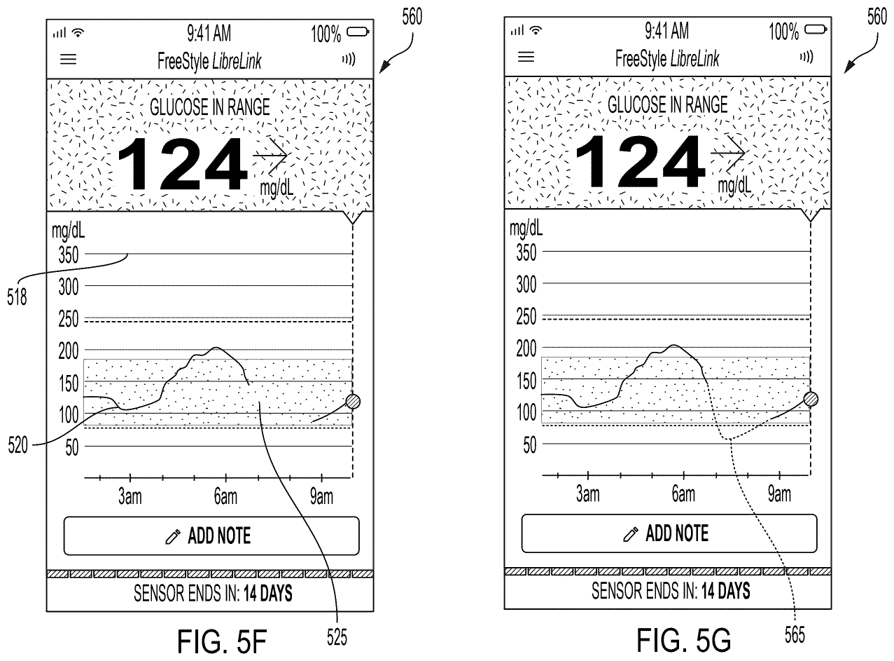

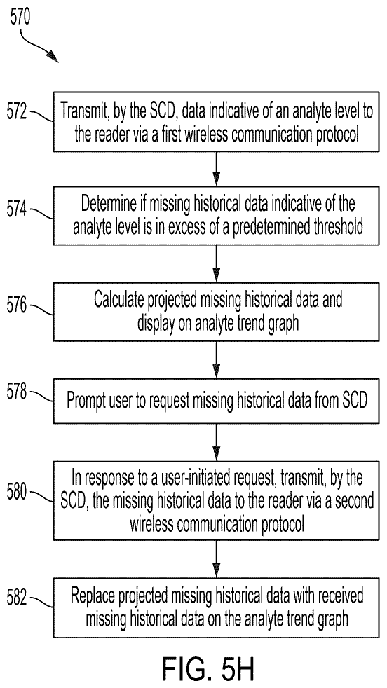

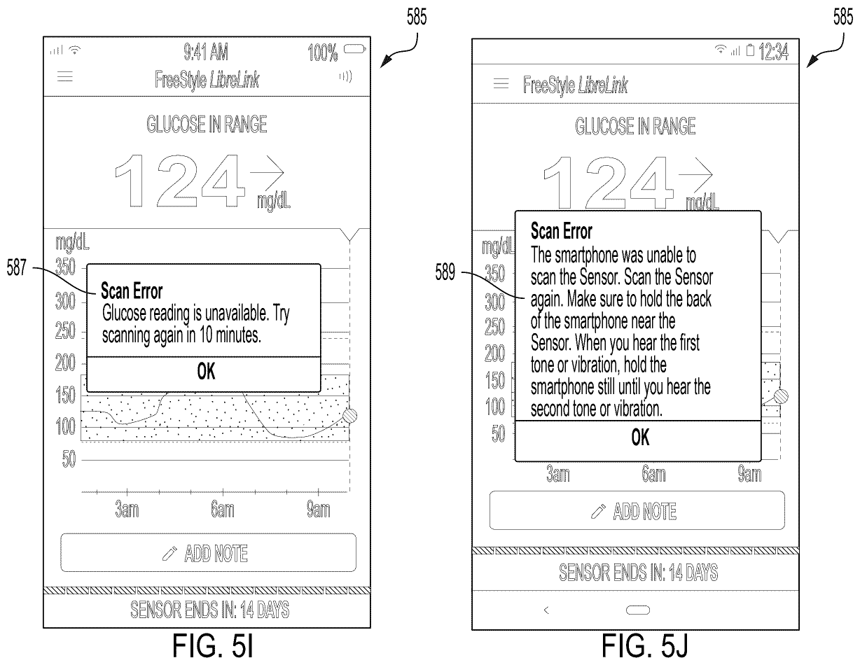

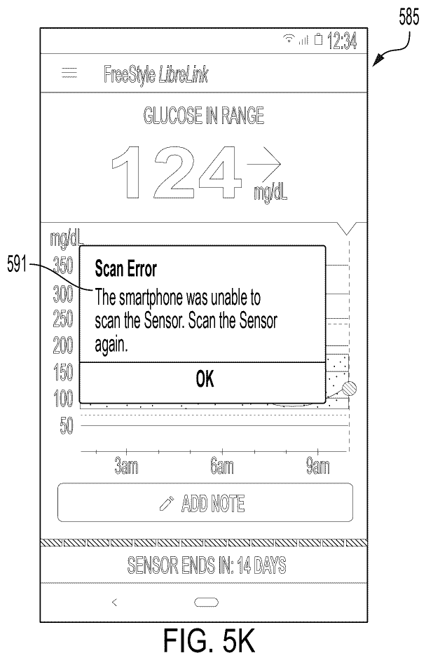

FIGS. 5A to 5D are example embodiments of GUIs for an analyte monitoring software application having a data backfilling feature, wherein the analyte monitoring software application is capable of communicating with a sensor control device according to more than one wireless communication protocol. According to one aspect of the example embodiments of FIGS. 5A to 5D, an analyte monitoring software application can be configured to receive data indicative of an analyte level of a user according to a first wireless communication protocol. Certain adverse conditions, however, can cause signal loss, signal degradation, latency, or other problems in the communication channel established between the sensor control device and the reader device using the first wireless communication protocol (e.g., Bluetooth or Bluetooth Low Energy). Consequently, these conditions can result in “gaps” in the historical analyte data displayed in the analyte monitoring software application.

According to the example embodiments of FIGS. 5A to 5D, the analyte monitoring software application can include an on-demand analyte data backfilling feature to obtain missing historical analyte data in order to fill the aforementioned gaps. According to one aspect of these example embodiments, the data backfilling feature can be configured to utilize a second wireless communication protocol that is different from the first wireless communication protocol. According to another aspect of these embodiments, the analyte monitoring software application can detect a gap in the historical analyte data and prompt the user to initiate the analyte data backfilling feature.

Referring to FIG. 5A, a home screen GUI 510 for an analyte monitoring software application is depicted, wherein home screen GUI 510 comprises an analyte trend graph section 518, including an analyte trend line 520. As seen in FIG. 5A, analyte trend line 520 includes a gap 525 in the data, perhaps due to an interruption in the communication channel established between the sensor control device and the reader device using the first wireless communication protocol.

Referring next to FIGS. 5B and 5C, the analyte monitoring software application can be configured to detect a gap 525 in analyte trend line 520. According to some embodiments, analyte monitoring software application can be configured to monitor for gaps in historical analyte data that exceed a predetermined threshold (e.g., more than two (2) hours of missing historical analyte data, more than three (3) hours of missing historical analyte data, or more than four (4) hours of missing historical analyte data). Those of skill in the art will understand that the example predetermined thresholds are illustrative only, and other thresholds can be implemented. In response to identifying a gap in excess of the predetermined threshold, the analyte monitoring software application can prompt the user to initiate a request for data indicative of the user's analyte level from the sensor control device via a second wireless communication protocol. In some embodiments, for example, the analyte monitoring software application can present an informational modal, such as modals 526 and 530 of FIGS. 5B and 4C, respectively, that instructs the user to actuate a scan icon to initiate the request for analyte data.

Referring next to FIG. 5D, home screen GUI 510 of analyte monitoring software application is depicted after receiving the missing historical analyte data from the sensor control device via the second wireless communication protocol. As can be seen in the analyte trend graph section of FIG. 5D, the analyte trend line includes a portion 535 reflecting the missing historical analyte data received from the sensor control device via the second wireless communication protocol. Further, in some embodiments, the analyte monitoring software application can also present a modal 532 indicating that the analyte data backfilling process is complete.

FIG. 5E depicts an example embodiment of a method 540 for backfilling analyte data in an analyte monitoring software application capable of receiving data indicative of an analyte level from a sensor control device according to more than one wireless communication protocol. In one aspect, method 540 can be a feature of an analyte monitoring software application stored in the memory of a reader device, which can be implemented with any one or more of the GUIs described with respect to FIGS. 5A to 5D, 5F, 5G, and 5I to 5L.

First, at Step 542, a sensor control device transmits data indicative of an analyte level to a reader device via a first wireless communication protocol. As described earlier, in many embodiments, the first wireless communication protocol can be a Bluetooth or Bluetooth Low Energy protocol. At Step 544, the analyte monitoring software application determines if there is missing historical data indicative of the analyte level in excess of a predetermined threshold. According to some embodiments, the predetermined threshold can be, for example, more than two (2) hours of missing historical analyte data, more than three (3) hours of missing historical analyte data, or more than four (4) hours of missing historical analyte data. Those of skill in the art will appreciate that other values can be utilized for the predetermined threshold. Additionally, in some embodiments, the predetermined threshold can be evaluated using consecutive points of missing historical analyte data. In other embodiments, the predetermined threshold can be evaluated using a cumulative number of points of missing historical analyte data, regardless of whether said points are consecutive or non-consecutive. In still other embodiments, the predetermined threshold can be evaluated using a cumulative number of points of missing historical analyte data within a sliding window of time.

Referring still to FIG. 5E, at Step 546, if it is determined that the predetermined threshold is met or exceeded, then the analyte monitoring software application can prompt the user to request the missing historical analyte data from the sensor control device via a second wireless communication protocol. In many embodiments, the user prompt can be in the form of a modal having instructions on how to initiate a scan of the sensor control device, as described with respect to FIGS. 5B and 5C.

At Step 548, in response to a user-initiated request, the sensor control device transmits the missing historical analyte data to the reader via the second wireless communication protocol. As described earlier, in many embodiments, the second wireless communication protocol can be an NFC protocol. Subsequently, at Step 550, the missing historical analyte data is received by the reader device via the second wireless communication protocol, and then displayed on the analyte trend graph in the analyte monitoring software application, as described with respect to FIG. 5D.

According to some embodiments, the analyte monitoring software application can also include a feature to calculate and display projected missing historical analyte data on the analyte trend graph. FIGS. 5F and 5G are example embodiments of GUIs for an analyte monitoring software application having a data backfilling feature that is capable of calculating and displaying projected missing historical analyte data. Referring to FIG. 5F, a home screen GUI 560 is depicted with an analyte trend graph section 518, including an analyte trend line 520. As seen in FIG. 5F, analyte trend line 520 includes a gap 525. According to some embodiments, if gap 525 exceeds a predetermined threshold for missing historical analyte data, then the analyte monitoring software application can be configured to calculate and display a projected missing historical analyte data 565 on the analyte trend graph, as shown in FIG. 5G. In some embodiments, the projected missing historical analyte data 565 can be displayed in a different color or line pattern to indicate that the indicated portion of the analyte trend line is based on projected data. According to some embodiments, the projected missing historical analyte data 565 can be calculated based on the user's previous historical analyte data and a pattern recognition algorithm. For example, the pattern recognition algorithm can be based on a function of one or more of: time-of-day, rate-of-change, rate of rate-of-change, and median glucose level, among other factors. In other embodiments, the projected missing historical analyte data 565 can be based on a regression analysis, a least squares method, and/or other forms of statistical modeling.

FIG. 5H depicts an example embodiment of a method 570 for displaying projected missing historical data in an analyte monitoring software application capable of receiving data indicative of an analyte level from a sensor control device according to more than one wireless communication protocol. At Step 572, a sensor control device transmits data indicative of an analyte level to a reader device via a first wireless communication protocol. As described earlier, in many embodiments, the first wireless communication protocol can be a Bluetooth or Bluetooth Low Energy protocol. At Step 574, the analyte monitoring software application determines if there is missing historical data indicative of the analyte level in excess of a predetermined threshold. According to some embodiments, the predetermined threshold can be, for example, more than two (2) hours of missing historical analyte data, more than three (3) hours of missing historical analyte data, or more than four (4) hours of missing historical analyte data. Those of skill in the art will appreciate that other values can be utilized for the predetermined threshold. Additionally, in some embodiments, the predetermined threshold can be evaluated using consecutive points of missing historical analyte data. In other embodiments, the predetermined threshold can be evaluated using a cumulative number of points of missing historical analyte data, regardless of whether said points are consecutive or non-consecutive. In still other embodiments, the predetermined threshold can be evaluated using a cumulative number of points of missing historical analyte data within a sliding window of time

Referring still to FIG. 5H, at Step 576, if it is determined that the predetermined threshold is met or exceeded, then the analyte monitoring software application can calculate projected missing historical data. In some embodiments, the projected missing historical analyte data can be calculated based on the user's previous historical analyte data and a pattern recognition algorithm. For example, the pattern recognition algorithm can include time-of-day, rate-of-change, rate of rate-of-change, and median glucose level, among other factors. In other embodiments, projected missing historical analyte data 565 can utilize regression analysis, least squares method, and/or other forms of statistical modeling.

According to another aspect of some embodiments, the projected missing historical analyte data can be displayed on the analyte trend graph along with the previous historical analyte data received via the first wireless communication protocol. As described with respect to FIG. 5G, the projected missing historical analyte data 565 can be displayed in a different color and/or line pattern to distinguish it from the previous historical analyte data 520.

Next, at Step 578, the analyte monitoring software application can prompt the user to request the missing historical analyte data from the sensor control device via a second wireless communication protocol. In many embodiments, the user prompt can be in the form of a modal having instructions on how to initiate a scan of the sensor control device, such as the embodiments depicted in FIGS. 5B and 5C.

At Step 580, in response to a user-initiated request, the sensor control device transmits the missing historical analyte data to the reader via the second wireless communication protocol. As described earlier, in many embodiments, the second wireless communication protocol can be an NFC protocol. Subsequently, at Step 582, the missing historical analyte data is received by the reader device via the second wireless communication protocol, and then displayed in the analyte monitoring software application on the analyte trend graph, as described with respect to FIG. 5D. According to one aspect of some embodiments, the received missing historical analyte data can replace the projected missing historical analyte data on the analyte trend graph.

In many embodiments, the analyte monitoring software application can also be configured to generate and display error messages relating to user-initiated requests for analyte data via the second wireless communication protocol. For example, FIGS. 5I to 5L depict example embodiments of GUIs for an analyte monitoring software application configured to generate and display error messages in response to failed attempts to request analyte data via the second wireless communication protocol. In some embodiments, for example, a modal 587 can be displayed on the screen indicating a “Scan Error,” and requesting that the user try to scan again in ten (10) minutes. In some embodiments, a modal 589 can be displayed with instructions to the user regarding how to position the reader device for initiating a scan request. In still other embodiments, a modal 591 can be displayed with instructions to the user to attempt to scan the sensor control device again.

### Example Embodiments of Systems, Methods, and GUIs for Enabling New Functionality in an Analyte Monitoring Software Application

Example embodiments of graphical user interfaces, methods and systems relating thereto for enabling a new functionality in an analyte monitoring software application will now be described. As an initial matter, it will be understood by those of skill in the art that any one or more of the example embodiments of the methods, interfaces, and systems described herein can either be implemented independently, or in combination with any of the other embodiments described in the present application. According to one aspect of the embodiments described herein, an analyte monitoring software application may occasionally receive a new feature or function, or an upgraded feature or function, such as the capability to receive data indicative of an analyte level from more than one wireless communication protocol, for example. In those scenarios, however, it can be disruptive to a user to enable a new feature or function (or an upgraded feature or function) of the analyte monitoring software application in the middle of a sensor wear. Therefore, it may be desirable to enable the new feature or function (or an upgraded feature or function) in between sensor wears.

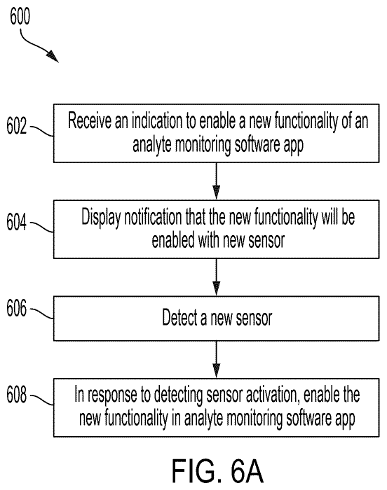

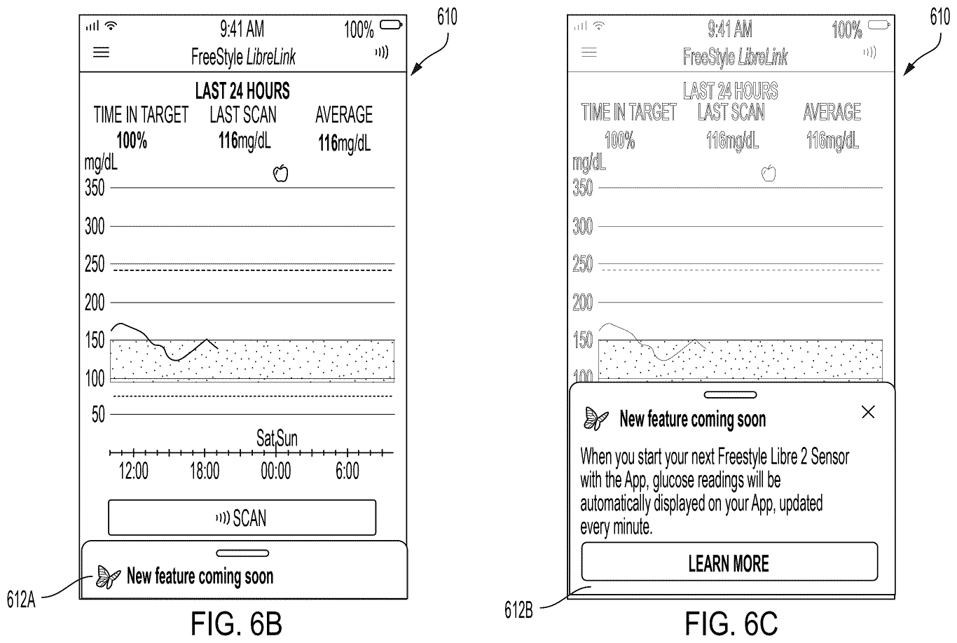

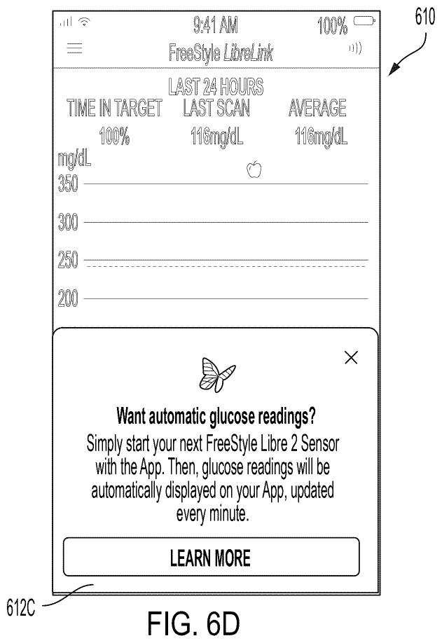

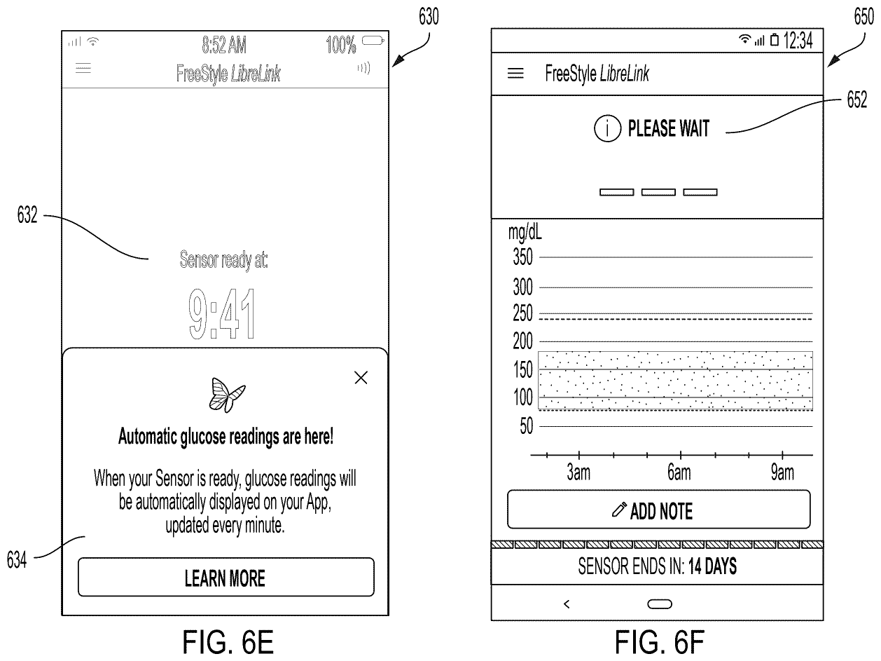

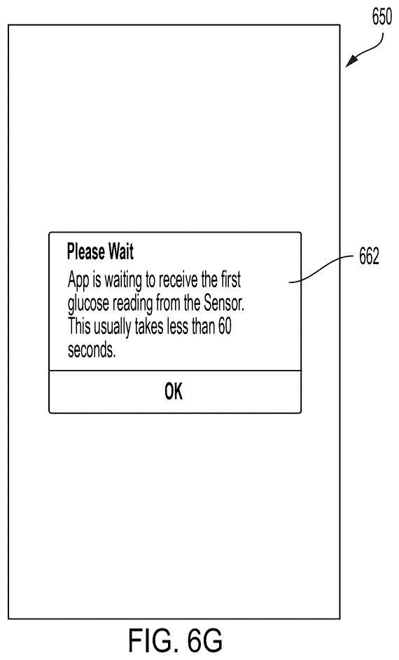

FIG. 6A depicts an example embodiment of a method 600 for enabling a new feature or functionality (or an upgraded feature or functionality) in an analyte monitoring software application. At Step 602, an indication to enable a new or upgraded feature or functionality is received by the analyte monitoring software application. In some embodiments, for example, this can occur when the analyte monitoring software application is updated through an enterprise app store, for example.

At Step 604, a notification is displayed to the user of the analyte monitoring software application regarding the new or upgraded feature or functionality. According to many embodiments, the notification can indicate to the user that the new or upgraded feature or functionality will be enabled with the next sensor.

Referring still to FIG. 6A, at Step 606, a new sensor is detected by the analyte monitoring software application. In some embodiments, for example, a new sensor can be detected when the old sensor has expired (i.e., the expiration time of the previous sensor has elapsed). In some embodiments, a new sensor can be detected when it is activated, e.g., by establishing a wireless communication link between a new sensor control device and the reader. In some embodiments, for example, the wireless communication link between the new sensor control device and the reader can be established during a Bluetooth or Bluetooth Low Energy pairing process. In other embodiments, the wireless communication link can be established during an NFC scanning process. In still other embodiments, a new sensor can be detected when a sensor code is inputted into the analyte monitoring software application by the user. Subsequently, at Step 608, in response to the detection of the new sensor, the new or upgraded feature or functionality is enabled in the analyte monitoring software application. In some embodiments, informational GUIs can be displayed to provide instructions to the user regarding how to use the new or upgraded feature or functionality.

FIGS. 6B to 6G are example embodiments of GUIs relating to the method 600 for enabling a new or upgraded feature or functionality in an analyte monitoring software application. Referring first to FIGS. 6B to 6D, in accordance with Step 602 of method 600, a home screen GUI 610 for an analyte monitoring software application is depicted, wherein one or more of modals 612A-C can be displayed to inform the user that a new feature will be enabled with the next sensor.

Referring next to FIG. 6E, in accordance with Steps 606 and 608, a sensor warm-up GUI 630 is depicted with a modal 634 to inform the user that a new feature has been enabled. Referring to FIGS. 6F and 6G, a new home screen GUI 650 is depicted after the new sensor has completed its warm up period. In some embodiments, a “Please Wait” notification and/or message 652 can be displayed on the new home screen GUI 650 for the first time after the new feature has been enabled, as there may not be previous historical data to be displayed. Similarly, in some embodiments, an informational modal 662 can be provided with the “Please Wait” notification and/or message 652 to provide further information to the user.

### Example Embodiments of Logbook Interfaces for an Analyte Monitoring Software App

Example embodiments of graphical user interfaces, methods, and systems relating thereto for a logbook feature of an analyte monitoring software application capable of receiving data indicative of an analyte level via more than one wireless communication protocol will now be described. As an initial matter, it will be understood by those of skill in the art that any one or more of the example embodiments of the methods, interfaces, and systems described herein can either be implemented independently, or in combination with any of the other embodiments described in the present application. According to some embodiments, the analyte monitoring software application may include a logbook feature for tracking events associated with the user's analyte data. In some embodiments, the events can be inputted by a user manually and can include, for example, events relating to meals, exercise, and/or medication. Furthermore, in some embodiments, the events can be automatically generated, and can include events relating to alarm conditions (e.g., an urgent low glucose alarm) or events generated by a connected device such as, for example, a wirelessly connected insulin pen.

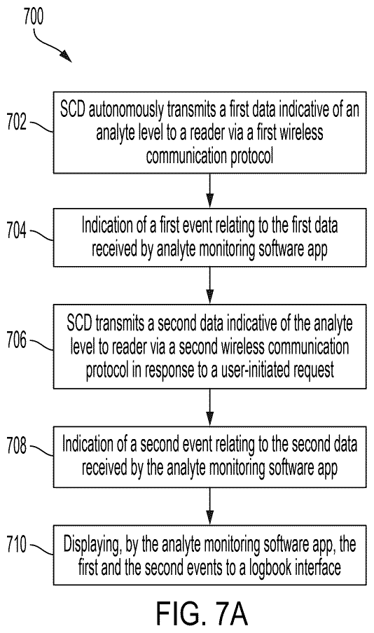

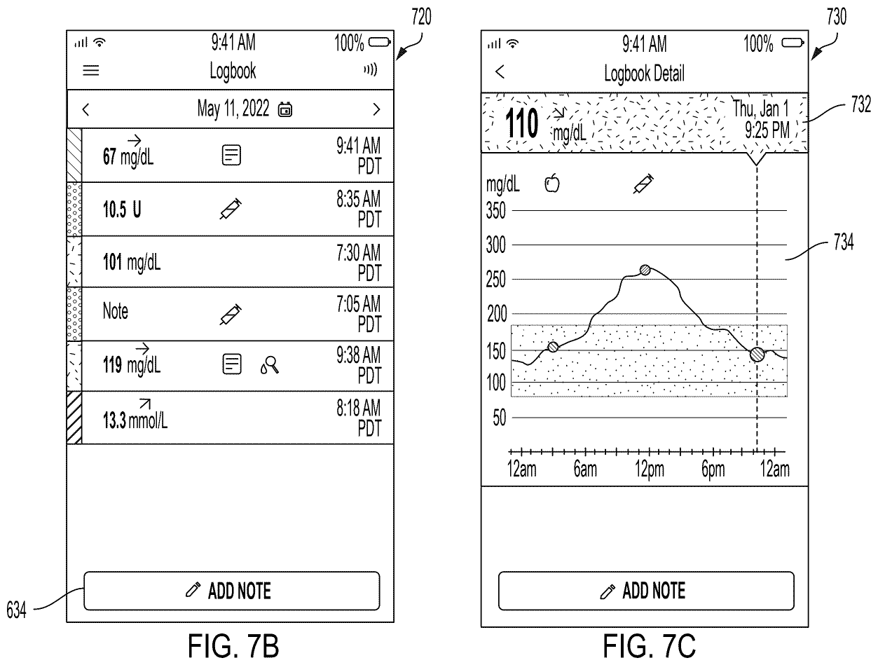

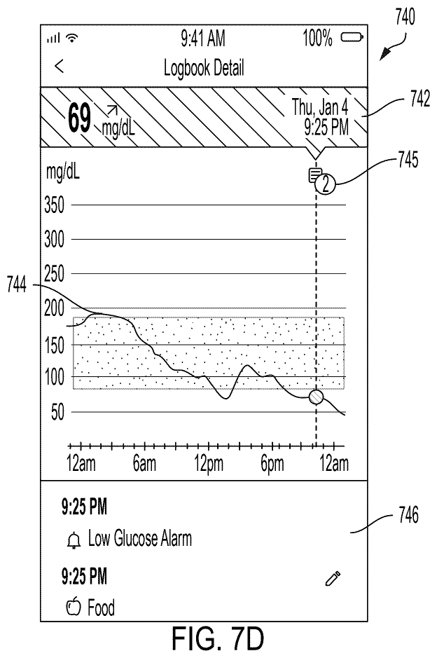

FIG. 7A depicts an example embodiment of a method 700 for displaying events in a logbook interface of an analyte monitoring software application capable of receiving data indicative of an analyte level according to more than one wireless communication protocol. Starting at Step 702, a sensor control device transmits a first data indicative of an analyte level to a reader device via a first wireless communication protocol. In some embodiments, the transmission can occur autonomously, e.g., without a request being sent from the reader to the sensor control device. For example, in some embodiments, as mentioned earlier, the first wireless communication protocol can be a Bluetooth or Bluetooth Low Energy protocol.

At Step 704, an indication of a first event relating to the first data indicative of the analyte level is received by the analyte monitoring software application. In some embodiments, the first event can be a manual entry or “note” created by the user (e.g., a meal, exercise, or medication event). In other embodiments, the first event can be automatically generated in response to an alarm condition being triggered (e.g., an urgent low glucose alarm).

At Step 706, the sensor control device transmits a second data indicative of the analyte level to the reader device via a second wireless communication protocol in response to a user-initiated request. In some embodiments, the user-initiated request can be, for example, the user actuating a scan button on a home screen GUI, as described with respect to FIGS. 4A to 4D. In other embodiments, the user-initiated request can be the user positioning the reader in close proximity to the sensor control device. In still other embodiments, the user-initiated request can be moving the reader according to a predetermined gesture or motion that can be detected by one or more motion sensors, positional sensors, or accelerometers disposed in the reader device. As described earlier, the second wireless communication protocol can be, for example, an NFC protocol.

Referring still to FIG. 7A, at Step 708, an indication of a second event relating to the second data indicative of the analyte level is received by the analyte monitoring software application. In some embodiments, the second event can be a current analyte level value that is automatically generated as a logbook entry when the user initiates a request for analyte data via the second wireless communication protocol (e.g., an NFC scan). In other embodiments, the second event can also be a manual entry created by the user (e.g., a meal, exercise, or medication event).

At Step 710, the first event relating to the first data indicative of the analyte level and the second event relating to the second data indicative of the analyte level can be displayed together in a single logbook interface, such as those described with respect to FIGS. 7B to 7D. In this manner, the analyte monitoring software application is capable of receiving and tracking multiple events in a single logbook interface, wherein the multiple events can relate to analyte data received through different wireless communication protocols.

FIGS. 7B to 7D are example embodiments of GUIs for an analyte monitoring software application having a logbook interface, and wherein the analyte monitoring software application is capable of communicating with a sensor control device according to more than one wireless communication protocol.

FIG. 7B is an example embodiment of a logbook GUI 720 containing multiple entries, including a first entry relating to a first data indicative of an analyte level received from the sensor control device via a first wireless communication protocol and a second entry relating to a second data indicative of an analyte level received from the sensor control device via a second wireless communication protocol. As described earlier, the multiple entries of logbook GUI 720 can include user inputted notes, medication dosage entries automatically generated and received from a connected insulin pen, entries relating to a user-initiated scan of the sensor control device, and/or alarm entries. Those of skill in the art will appreciate that the aforementioned entries are intended to be illustrative and non-exhaustive, and that logbook GUI 720 can include other types of entries and information.

FIGS. 7C and 7D are example embodiments of logbook detail GUIs 730 and 740. According to one aspect of some embodiments, logbook detail GUIs 730 and 740 can be accessed and displayed through logbook GUI 720 by selecting one of the entries. In some embodiments, the logbook detail GUI can comprise a banner section (732, 742) that includes a historical analyte level value along with a date and time stamp. In some embodiments, the logbook detail GUI can further include an analyte trend graph section (734, 744) that includes an analyte trend line. According to one aspect of some embodiments, a user can select any point along the analyte trend line of the analyte trend graph section (734, 744), to display a corresponding historical analyte level value and date and time stamp in banner section (732, 742).

Referring now to FIG. 7D, in some example embodiments, logbook detail GUI 740 can also comprise an event listing section 746, which can display a plurality of events for the logbook details GUI 740, wherein each event comprises an icon and a brief description. For example, as shown in FIG. 7D, the events listing section 746 indicates a low glucose alarm event and a food event at 9:25 PM. As shown at the top portion of FIG. 7D, a corresponding icon 745 for the low glucose alarm event and food event is shown as an icon adjacent to the analyte trend graph. In some embodiments, multiple events occurring at one time can be represented by a corresponding icon with a numbered badge or by a stacked icon.

### Example Embodiments of Methods for Dynamic Transmission Rate Features

Example embodiments of a dynamic transmission feature of an analyte monitoring software application, as well as systems and methods relating thereto, will now be described. According to one aspect of the embodiments of the present disclosure, an analyte monitoring software application capable of communicating with a sensor control device via more than one wireless communication protocol can result in more available and reliable analyte data for the user. However, an unintended consequence can be a significant increase in the amount of data being transmitted between the analyte monitoring software program and a trusted computer system, such as, for example, a cloud-based server.

According to some embodiments, the analyte monitoring software application can include a dynamic transmission rate feature that is capable of modifying the rate of data transmission between the reader device and a trusted computer system depending on whether certain conditions are present. For example, a caregiver may have their own remote analyte monitoring software application on their own reader device to monitor the primary user and receive analyte-related alerts. In those circumstances, since the caregiver's remote analyte monitoring software application receives analyte data from the trusted computer system (e.g., the cloud), it is necessary for the primary user's reader device to transmit data to the trusted computer system at a high rate to ensure that the caregiver's application also receives the analyte data in real-time or near real-time. Similarly, if a primary user's medication delivery device receives the primary user's analyte data from a trusted computer system in order to make therapy decisions, it is also necessary for the primary user's reader device to transmit data to the trusted computer system at a high rate to ensure that the medication delivery device is receiving timely and real-time analyte data. By contrast, if a primary user is not being monitored by a caregiver, or does not have any connected devices that rely upon data from a trusted computer system, then that primary user's reader device can be configured to transmit data to the trusted computer system at a lower rate.

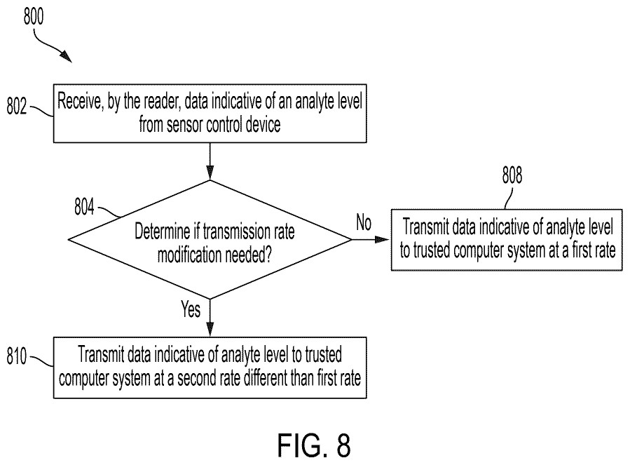

FIG. 8 depicts an example embodiment of a method 800 for a dynamic transmission rate feature of an analyte monitoring software application. At Step 802, the analyte monitoring software application on the reader device receives data indicative of an analyte level from the sensor control device. At Step 804, the analyte monitoring software application determines whether one of several transmission rate modification conditions are met. According to some embodiments, examples of transmission rate modification conditions can include whether the primary user is connected to a caregiver or health care provider that requires real-time monitoring of the primary user's analyte data, whether the primary user has a connected medication delivery device that requires real-time analyte data, and whether the primary user has manually set a transmission modification setting according to a predetermined schedule. The aforementioned list of conditions is not intended to be limiting, and those of skill in the art will appreciate that other transmission rate modification conditions can be implemented.

Referring still to FIG. 8, if none of the transmission rate modification conditions are met then, at Step 808, the analyte monitoring software application continues to transmit data indicative of the analyte level to a trusted computer system at a first rate. If one or more of the transmission rate modification conditions are met then, at Step 810, the analyte monitoring software application begin to transmit the data indicative of the analyte level to the trusted computer at a second rate that is different from the first rate. In some embodiments, for example, the transmission rate can be decreased if it is determined that the primary user is not being monitored by a caregiver, nor does the primary caregiver have any connected interfaces that require real-time or near real-time analyte data. In other embodiments, the transmission rate can be increased if it is determined that the primary user is now being monitored by a caregiver.

Although many of the embodiments described herein relate to glucose monitoring, those of skill in the art will appreciate that these same embodiments can be implemented for purposes of monitoring other analytes, such as, for example, lactate and ketones.

Furthermore, those of skill in the art will appreciate that the embodiments described herein are not limited to the monitoring one analyte at a time, although each embodiment described herein is capable of doing so. For example, according to some embodiments, a single sensor control device can include within its housing, for example, an analyte sensor capable of sensing an in vivo glucose level and an in vivo lactate level in a bodily fluid of the user. Likewise, any and all of the aforementioned embodiments of processes, display windows, methods, and/or alarms can be configured for purposes of monitoring multiple analytes at once (e.g., glucose and lactate, glucose and ketone, etc.).

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

Exemplary embodiments are set forth in the following numbered clauses:

1. An analyte monitoring system, comprising:
a sensor control device configured to be worn on skin of a subject, the sensor control device comprising an analyte sensor, wherein a portion of the analyte sensor is configured to be positioned through the skin and in fluid contact with a bodily fluid of the subject, and wherein the portion of the analyte sensor is further configured to sense an analyte level in the bodily fluid; and
a reader device, comprising:
   wireless communication circuitry configured to receive data indicative of the analyte level from the sensor control device; and
   one or more processors coupled with a memory, the memory storing an analyte monitoring software application,
wherein the sensor control device is configured to transmit a first data indicative of the analyte level to the reader device according to a first wireless communication protocol,
wherein the sensor control device is configured to transmit a second data indicative of the analyte level to the reader device according to a second wireless communication protocol in response to a user-initiated request, and
wherein the analyte monitoring software application, when executed by the one or more processors, further causes the one or more processors to output the first data indicative of the analyte level and the second data indicative of the analyte level to a single graphical user interface.

2. The analyte monitoring system of claim 1, wherein the first wireless communication protocol is a Bluetooth or Bluetooth Low Energy wireless communication protocol.

3. The analyte monitoring system of claim 1 or 2, wherein the second wireless communication protocol is a Near Field Communication protocol.

4. The analyte monitoring system of claim 1, 2 or 3, wherein the second wireless communication protocol is different from the first wireless communication protocol.

5. The analyte monitoring system of claim 1, 2, 3 or 4, wherein the single graphical user interface comprises an analyte trend graph comprising a first axis representative of time and a second axis representative of an analyte level concentration.

6. The analyte monitoring system of claim 5, wherein the analyte monitoring software application, when executed by the one or more processors, further causes the one or more processors to display a first portion of an analyte trend line on the analyte trend graph and a second portion of the analyte trend line on the analyte trend graph,
wherein the first portion of the analyte trend line is based on the first data indicative of the analyte level, and
wherein the second portion of the analyte trend line is based on the second data indicative of the analyte level.

7. The analyte monitoring system of any preceding claim, wherein the single graphical user interface includes a banner portion comprising a current glucose value and a trend arrow.

8. The analyte monitoring system of claim 7, wherein the analyte monitoring software program, when executed by the one or more processors, further causes the one or more processors to replace a first current glucose value based on the first data indicative of the analyte level with a second current glucose value based on the second data indicative of the analyte level.

9. A method, comprising:
receiving, by a reader device, a first data indicative of an analyte level from a sensor control device according to a first wireless communication protocol;
receiving, by the reader device, a second data indicative of the analyte level from the sensor control device according to a second wireless communication protocol; and
displaying, by an analyte monitoring software application installed on the reader device, the first data indicative of the analyte level and the second data indicative of the analyte level on a single graphical user interface,
wherein the second data indicative of the analyte level is transmitted by the sensor control device in response to a user-initiated request.

10. The method of claim 9, wherein the sensor control device comprises an analyte sensor, wherein a portion of the analyte sensor is configured to be positioned through the skin and in fluid contact with a bodily fluid of the subject, and wherein the portion of the analyte sensor is further configured to sense an analyte level in the bodily fluid.

11. The method of claim 9 or 10, wherein the first wireless communication protocol is a Bluetooth or Bluetooth Low Energy wireless communication protocol.

12. The method of claim 9, 10 or 11, wherein the second wireless communication protocol is a Near Field Communication protocol.

13. The method of claim 9, 10, 11 or 12, wherein the second wireless communication protocol is different from the first wireless communication protocol

14. The method of any of claims 9 to 13, wherein displaying the first data indicative of the analyte level and the second data indicative of the analyte level on the single graphical user interface comprises:
displaying a first portion of an analyte trend line on an analyte trend graph based on the first data indicative of the analyte level; and
displaying a second portion of the analyte trend line on the analyte trend graph based on the second data indicative of the analyte level.

15. The method of any of claims 9 to 14, wherein the single graphical user interface includes a banner portion comprising a current glucose value and a trend arrow.

16 The method of claim 15, wherein displaying the first data and the second data indicative of the analyte level on the single graphical user interface comprises replacing a first current glucose value based on the first data indicative of the analyte level with a second current glucose value based on the second data indicative of the analyte level.

17. A method, comprising:
transmitting, by a sensor control device, data indicative of an analyte level to a reader device via a first wireless communication protocol;
determining, by an analyte monitoring software application, whether there is missing historical data indicative of the analyte level which exceeds a predetermined threshold amount of data;
in response to determining that the predetermined threshold is exceeded, prompting a user within the analyte monitoring software application to request the missing historical data indicative of the analyte level;
in response to a user-initiated request, transmitting, by the sensor control device, the missing historical data indicative of the analyte level to the reader device via a second wireless communication protocol; and
displaying the missing historical data indicative of the analyte level on an analyte trend graph of the analyte monitoring software application.

18. The method of claim 17, wherein displaying the missing historical data indicative of the analyte level on the analyte trend graph comprises displaying the missing historical data adjacent to the data indicative of the analyte level transmitted via the first wireless communication protocol.

19. The method of claim 17 or 18, further comprising:
calculating projected missing historical data indicative of an analyte level; and
displaying the projected missing historical data indicative of the analyte level on the analyte trend graph of the analyte monitoring software application.

20. The method of claim 19, wherein the projected missing historical data is displayed in a color or line pattern different from an analyte trend line of the analyte trend graph based on the data indicative of the analyte level transmitted via the first wireless communication protocol.

21. The method of any of claims 17 to 20, wherein the first wireless communication protocol is a Bluetooth or Bluetooth Low Energy wireless communication protocol.

22. The method of any of claims 17 to 21, wherein the second wireless communication protocol is a Near Field Communication protocol.

23 The method of any of claims 17 to 22, wherein the second wireless communication protocol is different from the first wireless communication protocol.

24. A method, comprising:
receiving, at a reader device, an indication to enable a new functionality of an analyte monitoring software application;
displaying a notification indicating that the new functionality will be enabled with a new sensor;
detecting a new sensor; and
in response to the detection of the new sensor, enabling the new functionality in the analyte monitoring software application.

25. The method of claim 24, further comprising:
replacing a first home screen graphical user interface (GUI) with a second home screen GUI when the new functionality of the analyte monitoring software application is enabled.

26. The method of claim 24, wherein the notification is a modal.

27. The method of claim 24, further comprising:
displaying a modal indicating that the new functionality of the analyte monitoring software application has been enabled.

28. The method of claim 27, wherein the modal is displayed during a sensor warm-up period.

29. An analyte monitoring system, comprising:
a sensor control device configured to be worn on skin of a subject, the sensor control device comprising an analyte sensor, wherein a portion of the analyte sensor is configured to be positioned through the skin and in fluid contact with a bodily fluid of the subject, and wherein the portion of the analyte sensor is further configured to sense an analyte level in the bodily fluid; and
a reader device, comprising:
   wireless communication circuitry configured to receive data indicative of the analyte level from the sensor control device; and
   one or more processors coupled with a memory, the memory storing an analyte monitoring software application,
wherein the sensor control device is configured to transmit a first data indicative of the analyte level to the reader device according to a first wireless communication protocol,
wherein the sensor control device is configured to transmit a second data indicative of the analyte level to the reader device according to a second wireless communication protocol in response to a user-initiated request, and
wherein the analyte monitoring software application, when executed by the one or more processors, further causes the one or more processors to:
display a first event relating to the first data indicative of the analyte level and a second event relating to the second data indicative of the analyte level in a logbook interface of the analyte monitoring software application.

30. The analyte monitoring system of claim 29, wherein the first wireless communication protocol is a Bluetooth or Bluetooth Low Energy wireless communication protocol.

31. The analyte monitoring system of claim 29, wherein the second wireless communication protocol is a Near Field Communication protocol.

32. The analyte monitoring system of claim 29, wherein the second wireless communication protocol is different from the first wireless communication protocol.

33. The analyte monitoring system of claim 29, wherein at least one of the first event and the second event comprises a user inputted note relating to a meal.

34. The analyte monitoring system of claim 29, wherein at least one of the first event and the second event comprises a user inputted note relating to exercise.

35. The analyte monitoring system of claim 29, wherein at least one of the first event and the second event comprises a user inputted note relating to a medication dosage.

36. The analyte monitoring system of claim 29, wherein at least one of the first event and the second event comprises an automatically created entry based on an alarm.

37. The analyte monitoring system of claim 29, wherein at least one of the first event and the second event comprises an automatically created entry based on a medication dosage received from a connected insulin pen.

38. The analyte monitoring system of claim 29, wherein the second event comprises a current glucose level at the time of the user-initiated request.

39. A method, comprising:
receiving, by an analyte monitoring software application installed on a reader device, data indicative of an analyte level from a sensor control device;
determining, by the analyte monitoring software application, if a transmission rate modification condition is met; and
in response to a determination that the transmission rate modification condition is not met, transmitting, by the analyte monitoring software application, the data indicative of the analyte level to a trusted computer system at a first rate; and
in response to a determination that the transmission rate modification condition is met, transmitting, by the analyte monitoring software application, the data indicative of the analyte level to the trusted computer system at a second rate different than the first rate.

40. The method of claim 39, wherein the transmission rate modification condition comprises an indication that a primary user is being monitored by a caregiver.

41. The method of claim 39, wherein the transmission rate modification condition comprises an indication that a medication delivery device of a primary user is receiving the data indicative of the analyte level from the trusted computer system.

42. The method of claim 39, wherein the transmission rate modification condition comprises a user-configurable setting configured to modify the transmission rate according to a predetermined schedule.

43. The method of claim 39, wherein the transmission rate modification condition comprises an indication that a primary user is being monitored by a caregiver, and wherein the second rate is greater than the first rate.

44. The method of claim 39, wherein the trusted computer system is a cloud-based server platform.

1-16. (canceled)

17. A method, comprising:
transmitting, by a sensor control device, data indicative of an analyte level to a reader device via a first wireless communication protocol;
determining, by an analyte monitoring software application, whether there is missing historical data indicative of the analyte level which exceeds a predetermined threshold amount of data;
in response to determining that the predetermined threshold is exceeded, prompting a user within the analyte monitoring software application to request the missing historical data indicative of the analyte level;
in response to a user-initiated request, transmitting, by the sensor control device, the missing historical data indicative of the analyte level to the reader device via a second wireless communication protocol; and
displaying the missing historical data indicative of the analyte level on an analyte trend graph of the analyte monitoring software application.

18. The method of claim 17, wherein displaying the missing historical data indicative of the analyte level on the analyte trend graph comprises displaying the missing historical data adjacent to the data indicative of the analyte level transmitted via the first wireless communication protocol.

19. The method of claim 18, further comprising:
calculating projected missing historical data indicative of an analyte level; and
displaying the projected missing historical data indicative of the analyte level on the analyte trend graph of the analyte monitoring software application.

20. The method of claim 19, wherein the projected missing historical data is displayed in a color or line pattern different from an analyte trend line of the analyte trend graph based on the data indicative of the analyte level transmitted via the first wireless communication protocol.

21. The method of claim 17, wherein the first wireless communication protocol is a Bluetooth or Bluetooth Low Energy wireless communication protocol.

22. The method of claim 17, wherein the second wireless communication protocol is a Near Field Communication protocol.

23. The method of claim 17, wherein the second wireless communication protocol is different from the first wireless communication protocol.

24-44. (canceled)

45. The method of claim 17, wherein the missing historical data indicative of the analyte level is associated with a gap on the analyte trend graph of the analyte monitoring software application.

46. The method of claim 17, wherein the predetermined threshold comprises a predetermined number of hours of missing historical data indicative of the analyte level.

47. The method of claim 17, wherein the predetermined threshold comprises a predetermined cumulative number of consecutive or non-consecutive data points.

48. An analyte monitoring system, comprising:
a sensor control device configured to be worn on skin of a subject, the sensor control device comprising an analyte sensor, wherein a portion of the analyte sensor is configured to be positioned through the skin and in fluid contact with a bodily fluid of the subject, and wherein the portion of the analyte sensor is further configured to sense an analyte level in the bodily fluid,
wherein the sensor control device further comprises wireless communication circuitry of the sensor control device configured to communicate with a reader device via a first wireless communication protocol and a second wireless communication protocol; and
the reader device, comprising:
wireless communication circuitry of the reader device configured to receive the data indicative of the analyte level from the sensor control device via the first wireless communication protocol; and
one or more processors coupled with a memory, the memory storing an analyte monitoring software application,
wherein the analyte monitoring software application, when executed by the one or more processors, causes the one or more processors to:
determine whether there is missing historical data indicative of the analyte level which exceeds a predetermined threshold amount of data,
in response to a determination that the predetermined threshold is exceeded, prompt a user to request the missing historical data indicative of the analyte level, and
receive from the sensor control device via the second wireless communication protocol and display the missing historical data indicative of the analyte level on an analyte trend graph of the analyte monitoring software application, and
wherein the sensor control device is configured to transmit, in response to a user-initiated request, the missing historical data indicative of the analyte level to the reader device via the second wireless communication protocol.

49. The analyte monitoring system of claim 48, wherein the analyte monitoring software application, when executed by the one or more processors, further causes the one or more processors to:
display the missing historical data indicative of the analyte level adjacent to the data indicative of the analyte level transmitted via the first wireless communication protocol.

50. The analyte monitoring system of claim 49, wherein the analyte monitoring software application, when executed by the one or more processors, further causes the one or more processors to:
calculate projected missing historical data indicative of the analyte level; and
display the projected missing historical data indicative of the analyte level on the analyte trend graph of the analyte monitoring software application.

51. The analyte monitoring system of claim 50, wherein the projected missing historical data is displayed in a color or line pattern different from an analyte trend line of the analyte trend graph based on the data indicative of the analyte level transmitted via the first wireless communication protocol.

52. The analyte monitoring system of claim 48, wherein the first wireless communication protocol is a Bluetooth or Bluetooth Low Energy wireless communication protocol.

53. The analyte monitoring system of claim 48, wherein the second wireless communication protocol is a Near Field Communication protocol.

54. The analyte monitoring system of claim 48, wherein the second wireless communication protocol is different from the first wireless communication protocol.

55. The analyte monitoring system of claim 48, wherein the missing historical data indicative of the analyte level is associated with a gap on the analyte trend graph of the analyte monitoring software application.

56. The analyte monitoring system of claim 48, wherein the predetermined threshold comprises a predetermined number of hours of missing historical data indicative of the analyte level.

57. The analyte monitoring system of claim 48, wherein the predetermined threshold comprises a predetermined cumulative number of consecutive or non-consecutive data points.
